(19) United States
(12) Patent Application Publication
(10) Pub. No.: US 2007/0078448 A1
(43) Pub. Date: Apr. 5, 2007

(54) OCCLUSIVE WOUND DRESSING USEFUL IN TATTOO REMOVAL

(76) Inventor: Roger D.A. Lipman, Turnhout (BE)

Correspondence Address: Thomas W. Adams;Renner, Otto, Boisselle & Sklar, LLP, 19th Floor, 1621 Euclid Avenue, Cleveland, OH, 44115-2191, US

(21) Appl. No.: 11/468,961

(22) Filed: Aug. 31, 2006

Related U.S. Application Data
(63) Continuation of application No. PCT/US06/08361, filed on Mar. 9, 2006.
(60) Provisional application No. 60/665,029, filed on Mar. 24, 2005.

(51) Int. Cl. A61B 18/18

(52) U.S. Cl. 606/9

(57) ABSTRACT

Method of removing a tattoo from skin, including (a) applying laser radiation to a tattoo site; and (b) applying to the tattoo site a dressing to absorb pigment therefrom, the dressing including a fluid-absorbing pressure sensitive adhesive material. In one embodiment, the fluid-absorbing material includes an adhesive material and at least one hydrophilic polymer that is soluble and/or swellable in water. In one embodiment, the dressing includes a continuous phase and a discontinuous phase, the continuous phase including (b-1) one or more physically cross-linked solid rubber or (b-2) one or more styrene-containing thermoplastic elastomer or a mixture of (b-1) and (b-2), and the discontinuous phase including the one or more hydrophilic polymer that is soluble and/or swellable in water. In one embodiment, the one or more hydrophilic polymer includes at least one water-absorbent and/or water-swellable polymer, at least one hydrocolloid, or a mixture of two or more thereof.

### RELATED APPLICATIONS

This application is a continuation under 35 U.S.C. §120 of International Application No. PCT/US2006/008361, filed 09 Mar. 2006, which claims priority to U.S. Provisional Application No. 60/665,029, filed 24 Mar. 2005. The entire disclosure of this international application and the entire disclosure of this provisional application are hereby incorporated by reference.

### TECHNICAL FIELD

The present invention relates to an occlusive wound dressing useful in tattoo removal, e.g., from mammalian skin surfaces. In particular, the present invention relates to a fluid absorbing, pressure-sensitive adhesive paste composition for use in, e.g., laser tattoo removal procedures, in which use of the present invention results in accelerated pigment removal after treatment while promoting the healing of the skin surface from which the tattoo is removed.

### BACKGROUND

In the Western world, there are at least 50 million people with tattoos and many of these were applied when the wearer was young. At the present time, tattooing remains in vogue with people such as athletes and entertainers who, because they are role models for teenagers and young adults, continue to popularize the practice.

Concomitant with the increased popularity of tattooing is an increased need for effective tattoo removal. In later years, tattoos applied in the blush of youthful exuberance may no longer be acceptable to the wearer. Some estimate that as many as 50% of those who get tattoos later regret them. Tattoo removal using traditional methods has disadvantages such as incomplete pigment removal, non selective tissue destruction and unsatisfactory cosmetic results such as atrophic or hypertrophic scarring. Older methods of tattoo removal have involved the application of caustic chemicals such as phenol, sulfuric acid, tannic acid and zinc chloride. All of these methods are associated with a high incidence of scarring and pigmentation disturbances and are no longer used.

More refined methods of tattoo removal have included abrasion with salt (salabrasion), cryosurgery, dermabrasion, electrocoagulation, and the use of an infrared coagulator. All of these procedures are associated with significant scarring and the result after tattoo removal can appear worse than the tattoo alone. Conventional surgical methods have been used to treat tattoos however their use is limited to the removal of small tattoos. Surgical removal of large tattoos usually yields unacceptable results and it is no longer used.

The use of lasers to remove tattoos began in the early 1990s with the first report of successful tattoo removal using Q-Switched Ruby lasers. Ruby lasers are still quite useful for tattoo removal but they must be used carefully on individuals with a dark skin type since coincident melanin absorption at 694 nm can be associated with prolonged but usually temporary hypopigmentation in the treatment areas. In 1991 the Q-Switched Neodymium-YAG laser was introduced for tattoo removal. Theoretically, this laser with a longer wavelength would allow deeper penetration and at the same time exhibit less melanin absorption. Early studies using this laser showed that it was very effective in removing dark blue black ink seen in amateur and professional tattoos and especially useful in removing dark ink from cosmetic tattoos. The addition of a frequency doubling crystal to this laser, providing laser output at 532 nm or green light, allowed the removal of tattoos containing red and orange ink. At the present time the Q-Switched YAG laser is the most used laser for tattoo removal. Unfortunately, ink colors such as green do not respond to this laser necessitating the use of Ruby or Alexandrite lasers for complete removal. In 1992, a Q-Switched Alexandrite laser was introduced operating at a wavelength of 755 nm. The Alexandrite crystal emits energy that is longer than the Ruby at 694 nm and significantly shorter than the ND-YAG laser at 1064 nm. Excellent results have been obtained in the treatment of blue-black and green tattoos using this laser.

Lasers work by producing short pulses of intense light that pass harmlessly through the top layers of the skin to be selectively absorbed by the tattoo pigment. This laser energy causes the tattoo pigment to fragment into smaller particles that are then removed by the body's immune system. Researchers have determined which wavelengths of light to use and how to deliver the output of the laser effectively to remove tattoo ink. The laser does not affect normal skin pigment.

Treatment of the tattoo area using CO₂ or Erbium:YAG lasers is increasingly becoming the most popular way to achieve this removal. The thermal energy generated by the laser beam builds up steam in each cell and causes explosion of the individual cells, leading to freeing of the tattoo pigment and eventual liberation through the lymphatic system. Several treatments at intervals of about 6-8 weeks are necessary for complete removal of the tattoo in most cases. The resulting wound involves primarily the epidermis. The treatment creates an acute wound that takes one to two weeks to heal. It causes erythema, swelling, and oozing. There may be pain and tenderness with a possibility of (base) pigmentary alteration of the treated areas, infection and scarring.

The wound is currently treated by application of an antibiotic ointment and a dressing pad held in place with adhesive tape.

For these reasons, a need remains for a dressing that will decrease healing time, increase comfort for the patients and will accelerate pigment removal from the tattoo site.

### SUMMARY

The present invention relates to dressings for use in tattoo removal procedures. The presently disclosed dressings may decrease healing time, increase comfort for the patients and accelerate pigment removal from the tattoo site.

In one embodiment, the present invention relates to a method of removing a tattoo from skin, comprising:

(a) applying laser radiation to a tattoo site; and

(b) applying to the tattoo site a dressing to absorb pigment from the tattoo site, wherein the dressing comprises a fluid-absorbing pressure sensitive adhesive material. In one embodiment, the fluid-absorbing pressure sensitive adhesive material includes a mixture of an adhesive material and one or more hydrophilic polymer that is soluble and/or swellable in water. In one embodiment, the one or more hydrophilic polymer that is soluble and/or swellable in water comprises at least one water-absorbent and/or water-swellable polymer, at least one hydrocolloid, or a mixture of two or more thereof.

In one embodiment, the present invention relates to a method of removing a tattoo from skin, comprising:

(a) applying laser radiation to a tattoo site; and

(b) applying to the tattoo site a dressing to absorb pigment from the tattoo site, wherein the dressing comprises a continuous phase and a discontinuous phase, the continuous phase comprising (b-1) one or more physically cross-linked solid rubber or (b-2) one or more styrene-containing thermoplastic elastomer or a mixture of (b-1) and (b-2), and the discontinuous phase comprising one or more hydrophilic polymer that is soluble and/or swellable in water. Steps (a) and (b) may be repeated as needed.

In one embodiment, the continuous phase comprises the physically cross-linked solid rubber of (b-1), and further comprises one or more compatible liquid rubber, and one or more tackifier.

In one embodiment, the continuous phase comprises the physically cross-linked solid rubber of (b-1), which comprises a blend of linear or radial A-B-A block copolymers and up to about 85 wt % (wt %) of A-B block copolymer, based on the weight of the physically cross-linked solid rubber.

In one embodiment, the continuous phase comprises the one or more styrene-containing thermoplastic elastomer of (b-2), at least one compatible liquid rubber, polyisobutylene, and at least one oil.

In one embodiment, the discontinuous phase comprises from about 10 wt % to about 70 wt % of the total weight of the dressing.

In one embodiment, in the discontinuous phase the one or more hydrophilic polymer that is soluble and/or swellable in water comprises at least one water-absorbent and/or water-swellable polymer, at least one hydrocolloid, or a mixture of two or more thereof.

The dressing disclosed herein is generally pliable and in one embodiment contains no added ingredients that would irritate the skin in and around the tattoo site treated by the laser, so is comfortable to use. The dressing in the method of the present invention, may reduce the times required for removal of tattoo pigments and healing of the laser-treated tattoo site. Thus, the present invention addresses the need for a dressing that will decrease healing time, increase comfort for the patients and will accelerate pigment removal from the tattoo site.

### DETAILED DESCRIPTION

In one embodiment, the present invention relates to a method of removing a tattoo from skin, including steps of (a) applying laser radiation to a tattoo site; and (b) applying to the laser-applied tattoo site a dressing to absorb pigment from the tattoo site. The steps (a) and (b) may be repeated at suitable intervals, and step (b) may be repeated a plurality of times for each occurrence of (a).

In one embodiment, the dressing includes a fluid-absorbing pressure sensitive adhesive material. In one embodiment, the fluid-absorbing pressure sensitive adhesive material includes a mixture of an adhesive material and one or more hydrophilic polymer that is soluble and/or swellable in water. In one embodiment, the one or more hydrophilic polymer that is soluble and/or swellable in water comprises at least one water-absorbent and/or water-swellable polymer, at least one hydrocolloid, or a mixture of two or more thereof. In one embodiment, the dressing includes a pressure-sensitive adhesive hydrocolloid formulation including a dispersion of the fluid absorbing material in a pressure-sensitive adhesive matrix. The dispersion may be relatively uniform and/or may include a relatively continuous phase with a relatively discontinuous phase dispersed throughout the continuous phase. It will be recognized that in an embodiment including a relatively continuous phase and a relatively discontinuous phase, it may be difficult to discern boundaries between these phases, in the absence of some detectable, distinguishable feature (e.g., color) by which the phases can be distinguished. The dispersion of the materials may be sufficiently complete that it is not possible to distinguish continuous and discontinuous phases. Accordingly, in the following description, it is to be understood that in at least some embodiments, references to the continuous phase and the discontinuous phase may be more relevant to describing and defining the materials used to form the dressing than to describing or defining an actually observable physical difference in the resulting dressing itself.

In one embodiment, the dressing includes a continuous phase and a discontinuous phase. In one embodiment, the continuous phase includes either or both of (b-1) one or more physically cross-linked solid rubber or (b-2) one or more styrene-containing thermoplastic elastomer. In one embodiment, the discontinuous phase includes one or more hydrophilic polymer that is soluble and/or swellable in water. In one embodiment, the one or more hydrophilic polymer that is soluble and/or swellable in water includes at least one water-absorbent and/or water-swellable polymer, at least one hydrocolloid, or a mixture of two or more thereof.

The treatment method may further include applying one or more of an anesthetic agent, an antibiotic agent, an anti-infective agent, or a combination of two or more thereof to the tattoo site or to the patient systemically or both to the site and systemically. Application of such agents may take place before or after the steps (a) and/or (b). That is, for example, an anesthetic agent may be applied prior to step (a), and additional anesthetic agents may be applied after (a), either before or after (b) or between successive steps (b). Similarly, while it will be understood that an antibiotic agent and/or an anti-infective agent would often be applied between steps (a) and (b), these agents may be applied or the patient may be treated with these agents before step (a) or after step (b) or together with successive steps (b).

Continuous Phase Materials

Various embodiments of the continuous phase may be suitable for use in the present invention. Each of the various embodiments are discussed in the following, after which additional details relating to the specific materials used in each embodiment are provided. In addition to the materials disclosed in the following for each of the various embodiments of the continuous phase, the compositions may include any of the known additives useful in such compositions. The exact contents of a suitable dressing can be determined and adjusted by those of skill in the art, based upon the present disclosure and on the needs of the user and the person or patient undergoing the tattoo removal. For example, the contents of both the continuous and discontinuous phases may be suitably adjusted based on particular pigments used in the tattoo (if known), based on the particular body part or area from which the tattoo is to be removed, and/or based on factors such as skin type of the patient. In one embodiment, the continuous phase provides dry tack to adhere the adhesive to dry, i.e., not moist or wet, skin.

First Embodiment of Continuous Phase

In one embodiment of the continuous phase, the continuous phase comprises the physically cross-linked solid rubber of (b-1), and further comprises one or more compatible liquid rubber, and one or more tackifier.

In one embodiment, the cross-linked solid rubber includes at least one linear or radial A-B-A block copolymer based on styrene-butadiene, styrene isoprene or hydrogenated styrene-diene copolymers. These are commonly referred to as triblock copolymers, and are well known in the art.

In one embodiment, the cross-linked solid rubber comprises up to 85 wt %, based on the weight of the physically cross-linked solid rubber of one or more styrene-butadiene, styrene isoprene or hydrogenated styrene-diene A-B block copolymers. In one embodiment, the A-B block copolymer(s) are present in an amount of 15 to 80 wt %, based on the weight of the physically cross-linked solid rubber. In another embodiment, the A-B block copolymer(s) are present in an amount of 10 to 50 wt %, based on the weight of the physically cross-linked solid rubber. These A-B copolymers are commonly referred to as diblock copolymers, and are well known in the art. These block copolymers (both triblock and diblock) can be based on styrene-butadiene, styrene-isoprene, and hydrogenated styrene-diene copolymers such as styrene ethylene-butylene.

Suitable styrene-diene copolymers for this embodiment are exemplified by a blend of linear styrene-isoprene-styrene triblock copolymer and linear styrene-isoprene diblock copolymer, such as KRATON® D-1161 and KRATON® D-1117, both available from Kraton Polymers, Houston, Tex. Suitable hydrogenated styrene-diene copolymers are exemplified by a thermoplastic elastomer comprising a blend of clear linear triblock and diblock copolymer based on styrene and ethylene-butylene with a bound styrene of 13% mass, such as KRATON® G-1657 and a bound styrene of 30% mass, such as KRATON® G-1652. Further examples of such materials are provided below.

The liquid rubber component may include any suitable known liquid rubber. In one embodiment, the liquid rubber has a molecular weight of 25,000 to 50,000. In one embodiment, the liquid rubber has a glass transition temperature of less than −50° C. In one embodiment, the liquid rubber has a viscosity at 38° C. to 50 to 1000 Pas. Suitable liquid rubbers included, for example, KRATON® LVS1-101, an un-hydrogenated styrene-isoprene diblock copolymer having a molecular weight of 30,000 and a styrene content of 15%, and LIR-310, a styrene-isoprene copolymer having a molecular weight of about 31,000, available from Kuraray America, Pasadena, Tex.

In one embodiment, the weight ratio of the liquid rubber to the solid rubber is from 3:2 to 7:1. In another embodiment, the weight ratio of the liquid rubber to the solid rubber is from 1:1 to 5:1.

In one embodiment, the liquid rubber is other than, or in addition to, polyisobutylene. In some embodiments, polyisobutylene may also be included in the continuous phase, and when it is, in such an embodiment, it is in addition to the liquid rubber component.

Other materials may be added to the continuous phase to modify the properties for certain uses. Materials such as low molecular weight polybutenes, commercially available under the tradenames PARAPOL® 1300 (ExxonMobil) or HYVIS® 30 (BPAmoco), low molecular weight polyisobutylene, rubbers such as butyl rubber and high molecular weight polyisobutylene, mineral oil, and small amounts of other optional ingredients may be added. The addition of polymer stabilizers can be advantageous, to protect an unsaturated elastomer from degradation during processing. In one embodiment, the optional low molecular weight polybutenes may be added in amounts from 0% to about 20% of the weight of the continuous phase.

Other optional ingredients such as silica and optional active ingredients such as growth factors, antimicrobial compounds and wound-healing components such as collagen may also be incorporated into the compositions of this embodiment and of the other embodiments of the invention.

Second Embodiment of Continuous Phase

In one embodiment, continuous phase includes a physically cross-linked solid rubber comprising a blend of linear or radial A-B-A block copolymers and from 15 to 85 wt % of A-B block copolymer; a compatible tackifying resin; and a low-molecular weight polyisobutylene, in which the continuous phase is optionally modified by up to 50 wt % of butyl rubber.

Thus, in one embodiment, the continuous phase comprises the physically cross-linked solid rubber of (b-1), which comprises a blend of linear or radial A-B-A block copolymers and 15 to 85 wt % of A-B block copolymer, based on the weight of the physically cross-linked solid rubber, and further comprises the tackifying resin and low molecular weight PIB. Similar to the first embodiment of the continuous phase described above, in one embodiment, the A-B block copolymer(s) are present in an amount of 10 to 50 wt %, based on the weight of the physically cross-linked solid rubber. In one embodiment, the A-B-A block copolymer component of the cross-linked solid rubber comprises a styrene-olefin-styrene or styrene-alkane-styrene block copolymer. In one embodiment, the A-B-A block copolymer component of the solid rubber comprises a styrene-butadiene-styrene, styrene-isoprene-styrene or hydrogenated styrene-diene-styrene copolymer. In one embodiment, the A-B block copolymer component of the cross-linked solid rubber comprises a styrene-olefin or styrene-alkane (which may be a hydrogenated styrene-olefin) block copolymer. In one embodiment, the A-B block copolymer component of the solid rubber comprises a styrene-butadiene, styrene isoprene or hydrogenated styrene-diene copolymer. Suitable styrene-diene copolymers for use in this embodiment include, for example, KRATON® D-1161 and D-1117. Suitable hydrogenated styrene-diene copolymers for use in this embodiment include, for example, KRATON® G-1652 and G-1657. In addition, polymers in which there is a combination of chemically saturated blocks and chemically unsaturated blocks may be used. For example, one such material is a branched copolymer containing two polyisoprene chains attached to the rubber midblock of a styrene/ethylene-butylene/styrene triblock copolymer, available from Kraton as Research Product RP6919, with the trade name Tacky G. This material has a styrene content of 18%, and isoprene content of 36% and an ethylene-butylene content of 46 wt %. In another embodiment, a low styrene synthetic copolymer of butadiene and styrene, commonly referred to as SBR rubber, can be included as a solid rubber.

In one embodiment, the continuous phase contains from about 10 to about 30 wt % of the physically cross-linked solid rubber, based on the total weight of the continuous phase. In another embodiment, the continuous phase contains from about 15 to about 25 wt % of the physically cross-linked solid rubber, based on the total weight of the continuous phase.

In one embodiment, the continuous phase further comprises from about 18 to about 40 wt % of a compatible tackifying resin, based on the total weight of the continuous phase. In one embodiment, the weight ratio of solid rubber to tackifying resin is from about 1:0.5 to about 1:7. In another embodiment, the weight ratio of solid rubber to tackifying resin is from about 1:1 to about 1:5.

Suitable tackifiers include both naturally derived and synthetically produced tackifiers. The resins derived from α- and β-pinene such as PICCOLYTE® S-115, the pentaerythritol rosin esters such as PENTALYN® H, and trimethylol propane rosin esters such as STAYBELITE® Ester 10, are all useful in this and other embodiments the invention. Also cyclopentadienyl resins such as ESCOREZ® 5300, and ADTAC® LV-E, a C₅ synthetic hydrocarbon resin, are useful tackifiers in this and other embodiments the invention.

In one embodiment, the continuous phase further comprises from about 10 to about 60 wt % of a low-molecular weight polyisobutylene, based on the total weight of the continuous phase. In another embodiment, the continuous phase further comprises from about 20 to about 40 wt % of a low-molecular weight polyisobutylene, based on the total weight of the continuous phase. In one embodiment, the low-molecular weight polyisobutylene has a viscosity average molecular weight of about 30,000 to about 70,000. In another embodiment, the low-molecular weight polyisobutylene has a viscosity average molecular weight of about 40,000 to about 60,000. Suitable polyisobutylenes are commercially available, for example, the VISTANEX® LM series, available from ExxonMobil Chemical (from BASF in the future).

In one embodiment, the liquid rubber is other than, or in addition to, polyisobutylene. In some embodiments, polyisobutylene may also be included in the continuous phase, and when it is, in such an embodiment, it is in addition to the liquid rubber component.

In one embodiment, an elastomeric polymer such as butyl rubber or a high molecular weight polyisobutylene may be blended into the continuous phase. The high MW PIB or butyl rubber may be used in the viscosity average molecular weight range of about 200,000 to about 600,000 or, in another embodiment, a viscosity average molecular weight in the range from about 250,000 to about 400,000, and is exemplified by the grade Butyl 065. This and other grades of butyl rubber are available from ExxonMobil Chemical. The high molecular weight butyl rubber may be added in amount suitable to modify various properties of the final formulation, and may be from 0% to about 50% of the total weight of the continuous phase, typically 10 to 30 weight %, based on the total weight of the continuous phase. In another embodiment, the continuous phase further includes up to about 25 wt % of the modifying butyl rubber. In another embodiment, the continuous phase further includes up to about 10 wt % of the modifying butyl rubber.

Other materials may be added to the continuous phase to modify the properties for certain uses. Materials such as low molecular weight polybutenes, commercially available under the tradenames PARAPOL® 1300 (ExxonMobil) or HYVIS® 30 (BP/Amoco), rubbers such as high molecular weight polyisobutylene, mineral oil, and small amounts of other optional ingredients may be added. The addition of polymer stabilizers can be advantageous, to protect an unsaturated elastomer from degradation during processing. In one embodiment, the optional low molecular weight polybutenes may be added in amounts from 0% to about 20% of the weight of the continuous phase.

Other optional ingredients such as silica and optional active ingredients such as growth factors, antimicrobial compounds and wound-healing components such as collagen may also be incorporated into the compositions of this embodiment and of the other embodiments of the invention.

Third Embodiment of Continuous Phase

In one embodiment of the continuous phase, the continuous phase includes the one or more styrene-containing thermoplastic elastomer of (b-2), and further includes at least one compatible liquid rubber, at least one polyisobutylene, and at least one oil.

In one embodiment, the dressing includes the styrene-containing block copolymer, present in a range from about 1 wt % to about 9 wt % of the dressing composition; the liquid rubber, present in a range from about 0.5 wt % to about 9 wt % of the continuous phase; a polyisobutylene, present from about 15 wt % to about 40 wt % of the dressing composition; and a mineral oil component, present from about 25 wt % to about 45 wt % of the dressing composition, and at least one water-soluble and/or water-swellable absorbent polymer, present in an amount from about 25 wt % to about 55 wt % of the dressing composition.

In one embodiment, the dressing includes the styrene-containing block copolymer, present in a range from about 2 wt % to about 7 wt % of the dressing, and in another embodiment, from about 3 wt % to about 6 wt % of the dressing.

In one embodiment, the solid rubber comprises at least one linear or radial A-B-A block copolymer based on styrene-butadiene, styrene isoprene or hydrogenated styrene-diene copolymers.

In one embodiment, the continuous phase comprises up to about 85 wt %, based on the weight of the styrene-containing thermoplastic elastomer, of one or more styrene-butadiene, styrene isoprene or hydrogenated styrene-diene A-B block copolymers. In another embodiment, the continuous phase comprises from about 10 to about 50 wt %, based on the weight of the styrene-containing thermoplastic elastomer, of one or more styrene-butadiene, styrene isoprene or hydrogenated styrene-diene A-B block copolymers. Suitable styrene-diene copolymers for use in this embodiment include, for example, KRATON® D-1161 and D-1117. Suitable hydrogenated styrene-diene copolymers for use in this embodiment include, for example, KRATON® G-1652 and G-1657. In addition, polymers in which there is a combination of chemically saturated blocks and chemically unsaturated blocks. For example, one such material is a branched copolymer containing two polyisoprene chains attached to the rubber midblock of a styrene/ethylene-butylene/styrene triblock copolymer, available from Kraton as Research Product RP6919, with the trade name Tacky G. This material has a styrene content of 18%, and isoprene content of 36% and an ethylene-butylene content of 46 wt %. In another embodiment, a low styrene synthetic copolymer of butadiene and styrene, commonly referred to as SBR rubber, can be included as a solid rubber.

In one embodiment, the liquid rubber has a molecular weight in the range of about 25,000 to about 50,000, and in another embodiment, from about 30,000 to about 45,000. In one embodiment, the liquid rubber has a glass transition temperature of less than 50° C. In one embodiment, the liquid rubber has a viscosity at 38° C. in the range of about 500 to about 10,000 poises, and in one embodiment, from about 1000 to about 5000 poises. In one embodiment, the liquid rubber component comprises a low molecular weight liquid rubber. In one embodiment, the liquid rubber component has a number average molecular weight less than about 3,000.

In one embodiment, the amount of liquid rubber ranges up to about 9 wt % of the continuous phase. In one embodiment, the maximum amount of liquid rubber is limited to about 5 wt % of the continuous phase. In one embodiment, the amount of liquid rubber ranges from about 0.5 wt % to about 8 wt % of the continuous phase.

In one embodiment, the liquid rubber comprises at least one of synthetic liquid isoprene rubber, depolymerized natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer.

In one embodiment, the dressing includes a polyisobutylene, present from about 20 wt % to about 35 wt % of the dressing, and in another embodiment, from about 25 wt % to about 30 wt % of the dressing. In one embodiment, the content of the polyisobutylene is from about 15 wt % to about 40 wt % of the continuous phase. In another embodiment, the content of the polyisobutylene is from about 20% to about 30 wt % of the continuous phase.

In one embodiment, the polyisobutylene has a Flory viscosity average molecular weight in the range from about 25,000 to about 75,000. In another embodiment, the polyisobutylene has a Flory viscosity average molecular weight in the range from about 35,000 to about 55,000. In one embodiment, the polyisobutylene component is exemplified by the VISTANEX® LM series of polyisobutylenes, available from ExxonMobil Chemical Corporation (BASF in future).

In one embodiment, the liquid rubber is other than, or in addition to, polyisobutylene. In some embodiments, polyisobutylene may also be included in the continuous phase, and when it is, in such an embodiment, it is in addition to the liquid rubber component.

In one embodiment, the oil component is present in the adhesive paste composition within the range of about 10 wt % to about 45 wt % of the dressing, and in one embodiment, from about 15 wt % to about 40 wt % of the dressing.

In one embodiment, the oil component comprises mineral oil. In one embodiment, a portion of the mineral oil may be replaced by a liquid hydrocarbon or liquid polymeric material or by a natural vegetable oil. In one embodiment, the mineral oil is exemplified by the KAYDOL® series of materials from Witco Chemical. Witco White Mineral Oil USP has a viscosity at 40° C. between about 60 and about 75 mm²/s, and in one embodiment, between about 63 and about 70 mm²/s, as determined by test method ASTM D-445. However, any suitable mineral oil may be used. Mineral oil may also be referred to as liquid petrolatum, mineral spirits, adepsine oil, alboline, glymol, liquid paraffin, paraffin oil or saxol, some of which may be used as trade names. In one embodiment, the mineral oil has a boiling point in the range from about 179° C. to about 210° C.

In one embodiment, the oil component may comprise corn oil soybean oil, cottonseed oil, castor bean oil, palm oil, coconut oil, sunflower seed oil, canola oil, other known vegetable oils, animal oils such as fish oil, lard and tallow, and may further comprise synthetic triglycerides.

In one embodiment, the continuous phase further comprises at least one low molecular weight polybutene. In one embodiment, the optional low molecular weight polybutenes may be added in amounts from 0% to about 20% of the weight of the continuous phase. The low molecular weight polybutene, in one embodiment, has a molecular weight in the range from about 1000 to about 3000.

In one embodiment, the weight of the combination of the styrene-containing thermoplastic elastomer and the liquid rubber is the range of about 1% to about 9%, based on the total weight of the dressing. In one embodiment, the weight of the combination of the styrene-containing thermoplastic elastomer and the liquid rubber is the range of about 1.5% to about 5%, based on the total weight of the dressing.

Discontinuous Phase

As described above, the dressing comprises one or more hydrophilic polymer that is soluble and/or swellable in water. In one embodiment, the one or more hydrophilic polymer that is soluble and/or swellable in water is dispersed throughout the dressing with the pressure-sensitive adhesive material. In one embodiment, the one or more hydrophilic polymer that is soluble and/or swellable in water may be present in the dressing as a discontinuous phase, dispersed in a continuous phase formed by a pressure-sensitive adhesive as described above.

In one embodiment, the dressing comprises a discontinuous phase constituting from about 10 wt % to about 70 wt % of the total weight of the dressing. In another embodiment, the discontinuous phase comprises from about 20 wt % to about 50 wt % of the total weight of the dressing. In another embodiment, the discontinuous phase comprises from about 15 wt % to about 55 wt % of the dressing. Suitable relative quantities of the continuous and discontinuous phases can be determined by those of skill in the art based on the present disclosure and on the particular needs of the user of the dressing.

In one embodiment, the discontinuous phase comprises one or more hydrophilic polymer that is soluble and/or swellable in water. In one embodiment, the discontinuous phase comprises at least one water-absorbent and/or water-swellable polymer, at least one hydrocolloid, or a mixture of two or more thereof. The mixture may comprise one or more of each of both the water-absorbent and swellable polymer and the hydrocolloid, or a plurality of either the water-absorbent and water-swellable polymer or the hydrocolloid. The polymer and the hydrocolloid may be soluble or insoluble.

In one embodiment, the water-soluble and/or water-swellable polymer comprises about 25% to about 55 wt % of the total weight of the dressing. In one embodiment, the water-soluble and/or water-swellable polymer constitutes about 30 to about 50 wt % of the dressing, and in another embodiment, the water-soluble and/or water-swellable polymer constitutes from about 35 to about 45 wt % of the dressing.

In one embodiment, the discontinuous phase comprises one or more hydrocolloids that are soluble and/or swellable in water. The water-soluble and/or water-swellable hydrocolloids may be comprised of any combination of soluble and/or insoluble absorbents. The soluble hydrocolloids assist in adhering the dressing to moist body surfaces, and is known as “wet tack”. One or more water-swellable hydrocolloids may also be present. The hydrocolloids may be present in addition to the one or more hydrophilic polymer that is soluble and/or swellable in water, or may be that polymer.

In one embodiment, the at least one water-absorbent and/or water-swellable polymer comprises at least one of cross-linked sodium carboxymethyl cellulose, crystalline sodium carboxymethyl cellulose, cross-linked dextran, calcium alginate, starch-acrylonitrile graft copolymer, starch sodium polyacrylate, gluten, polymers of methylvinyl ether and maleic acid and derivatives thereof. The swellable polymer may also be a so-called “super absorbent” material such as starch sodium polyacrylate. Other hydratable polymers such as gluten and polymers of methyl vinyl ether and maleic acid and derivatives thereof may also be included in the discontinuous phase. One or more such polymers may be present and a mixture of soluble and insoluble polymers can be used.

In one embodiment, the water-soluble and/or water-swellable polymer may comprise at least one of starches such as flour starch, corn starch, potato starch, etc. In another embodiment, the water-soluble and/or water-swellable polymer may comprise mannan, such as yeast gum, manna or konjak. In another embodiment, the water-soluble and/or water-swellable polymer may comprise at least one of various seaweeds such as agar-agar, sodium alginate, etc. In another embodiment, the water-soluble and/or water-swellable polymer may comprise at least one plant mucilage such as tragacanth gum, gum arabic, karaya gum, guar gum, psyllium seed gum, dammar gum, pectin etc., and/or various proteins such as gelatin, collagen, casein, etc. In another embodiment, the water-soluble and/or water-swellable polymer may comprise at least one cellulose-derived material such as carboxymethyl cellulose, hydroxyethyl cellulose, methyl cellulose, etc., modified starches such as soluble starch, carboxymethyl starch, dialdehyde starch, a cross-linked dextrin, etc. In another embodiment, the water-soluble and/or water-swellable polymer may comprise at least one copolymer of starch or cellulose, such as starch-acrylonitrile graft copolymer, a starch polyacrylate salt. In another embodiment, the water-soluble and/or water-swellable polymer may comprise at least one synthetic resin such as polyvinyl alcohol, sodium polyacrylate, polyethylene oxide, etc., and copolymers of starches or celluloses and acrylonitrile, acrylic acid, methacrylic acid, vinyl alcohol, vinyl chloride, etc. In one embodiment, the water-soluble and/or water-swellable polymer may comprise at least one of plant mucilage such as tragacanth gum, gum arabic, karaya gum, guar gum, psyllium seed gum, dammar gum, pectin, etc., the celluloses such as CMC (carboxymethyl cellulose), HEC (hydroxyethyl cellulose), etc., and the copolymers of starches or celluloses and acrylonitrile, acrylic acid, sulfuric acid, vinyl sulfonate, etc. The foregoing embodiments may be combined with one another, and may be combined with water-swellable polymers and/or super-absorbent materials.

In one embodiment, the water-swellable polymers include, for example, hydroxypropylcellulose (HPC) and polyethylene oxide (PEO). HPC is available from commercial suppliers including, for example, Aqualon, Inc., (Wilmington, Del.). The useful HPC generally has an average molecular weight in the range of about 60,000 to 1,200,000. In another embodiment, the water-swellable polymer includes homopolymers and copolymers of carboxymethyl cellulose, hydroxyethyl cellulose, hydroxymethyl cellulose. In another embodiment, the water-swellable polymer includes a water-soluble or water-swellable polymer derived from acrylic acid or a pharmaceutically acceptable salt thereof, such as the polyacrylic acid polymers as follows: Polycarbophil (Noveon AA-1), carbomer (CARBOPOL® 974P or 971P or 907), or a water-soluble salt of a co-polymer of methyl vinyl ether and maleic acid or anhydride (Gantrez MS-955).

In one embodiment, the at least one hydrocolloid comprises at least one of sodium carboxymethyl cellulose, pectin, gelatin, guar gum, locust bean gum, collagen, gum arabic, karaya gum, alginic acid, sodium alginates, sodium-calcium alginates, calcium alginates, polyvinyl alcohol, polyvinyl pyrrolidone, polyethylene glycol and polypropylene glycol, and high molecular weight polyethylene glycols and polypropylene glycols.

In one embodiment, the hydrophilic polymer functions as the absorbent, and to provide “wet tack” that ensures the dressing adheres to the skin. In one embodiment, the hydrophilic polymer is capable of swelling in water and transporting water.

In one embodiment, fumed silica is useful as an additional optional additive. Fumed silica, such as AEROSIL® 200 manufactured by Degussa, can help in increasing the shear strength of the continuous phase. Some hydrocolloid adhesives have a propensity to cold flow. Cold flow is a measure of the viscous deformation of the adhesive under load which is manifested in the ability of the adhesive to squeeze out from under the backing or dressing. This is usually deleterious to dressing and barrier performance and the presence of silica can often improve cold flow performance.

Other components which may be added in minor amounts include pH controllers, bactericides, growth factors, wound healing components such as collagen and pigments such as titanium dioxide, TiO₂.

Additional Details on Certain Materials in Continuous Phase Embodiments

The styrenic components may include block or radial copolymers based on styrene-butadiene, styrene-isoprene or styrene ethylene-butylene. In addition, a low styrene synthetic copolymer of butadiene and styrene, commonly called SBR rubber, can be used as the thermoplastic elastomer. The elastomer may comprise linear or radial A-B-A block copolymers or mixtures of these A-B-A copolymers with simple A-B block copolymers. In one embodiment, the proportion of A-B block copolymers in the mixture of A-B-A and A-B block copolymers does not exceed about 85 wt %, and in other embodiments, lower percentages are used. In one embodiment, the proportion of A-B block copolymers in the mixture of A-B-A and A-B block copolymers does not exceed about 65 wt %, in another embodiment the proportion of A-B block copolymers in the mixture of A-B-A and A-B block copolymers does not exceed about 50 wt %, and in another embodiment, the proportion of A-B block copolymers in the mixture of A-B-A and A-B block copolymers does not exceed about 35 wt %, and in yet another embodiment, the proportion of A-B block copolymers in the mixture of A-B-A and A-B block copolymers does not exceed about 20 wt %.

In one embodiment, the elastomeric component comprises linear or radial A-B-A block copolymers or mixtures of these linear or radial A-B-A block copolymers with simple A-B block copolymers. In these block copolymers the A-blocks are derived from styrene or styrene homologs and the B-blocks are derived from conjugated dienes or lower alkenes.

The A-B-A block copolymers are of the type which consist of A blocks derived from styrene or one of its homologs and B blocks derived from conjugated dienes, such as butadiene or isoprene, or from lower alkenes such as ethylene or butylene. The radial A-B-A polymers useful in this embodiment are of the type described, for example, in U.S. Pat. No. 3,281,383 and conform to the general formula (A-B)ₙX, where A and B comprise blocks derived from the monomers described above in connection with the A-B-A copolymers, X is an organic or inorganic connecting moiety having a functionality of at least 2, and n is equal to the functionality of X. Homologs of styrene may include any known homolog of styrene for use in such A-B-A, A-B or (A-B)ₙX copolymers. For example, (C₁—C₈) alkylstyrenes such as—methylstyrene, o-, m- and p-methylstyrenes, o-, m- and p-ethylstyrene, 2,4-dimethylstyrene, p-n-butylstyrene, p-t-butylstyrene, p-n-hexylstyrene, p-n-octylstyrene, p-n-nonylstyrene and p-n-decylstyrene, arylstyrenes, such as p-phenylstyrene), alkoxy-substituted styrenes (such as p-methoxystyrene), hydroxyl-substituted styrenes (such as p-hydroxystyrene), halogen-substituted styrenes (such as p-chlorostyrene and 3,4-dichlorostyrene) and mixtures of two or more of these (such as mixtures of styrene with at least one substituted styrenes) may be suitable styrene homologs. As used herein the term “styrene” includes homologs thereof. These examples are intended to be non-limiting.

Suitable styrene-diene copolymers are exemplified by a blend of linear styrene-isoprene-styrene triblock copolymer and linear styrene-isoprene diblock copolymer. Such a material is available from Kraton Polymers, Houston, Tex. as KRATON® D-1161 N (referred to as D-1161 in Europe) and has a bound styrene content of about 15% and a diblock content of 17%. A second example is a blend of linear styrene-isoprene-styrene triblock copolymer and linear styrene-isoprene diblock copolymer available from Shell Chemical as KRATON® D-1117P and which has a bound styrene content of about 17% and a diblock content of 33%.

An example of a suitable hydrogenated styrene-diene copolymer is a thermoplastic elastomer comprising a blend of linear triblock and diblock copolymer based on styrene and ethylene-butylene with a bound styrene of about 14 wt %. Such a material is commercially available from Kraton Polymers as KRATON® G-1657M, which has a bound styrene content of about 13 wt %. Another example is KRATON® G-1652 (referred to as G-1652E in Europe) from Kraton Polymers, which is a thermoplastic elastomer comprised of a linear triblock copolymer based on styrene and ethylene-butylene, S-E/B-S, with a bound styrene content of about 30 wt %. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material is available from Kraton Polymers as KRATON® Research Product RP6919. This material has a styrene content of 18 wt % an isoprene content of 36 wt % and an ethylene-butylene content of 46 wt %.

In one embodiment, the continuous phase component is comprised of the thermoplastic elastomer and the liquid rubber component. In one embodiment, the continuous phase is substantially resin-free.

Liquid rubbers useful in this embodiment are synthetic liquid isoprene rubber, depolymerized natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer. In one embodiment, the liquid rubbers have a molecular weight in a range from about 2500 to about 50,000. In one embodiment, the liquid rubbers have a glass transition temperature of less than about 50° C., and a melt viscosity at 38° C. in the range from about 500 to about 10,000 poises.

It will be appreciated that other liquid rubbers known in the art could be useful in this embodiment of the present invention.

In one embodiment, the thermoplastic elastomer is a block copolymer of styrene and isoprene having a styrene content of about 13 wt % and an isoprene content of about 87 wt %, a glass transition temperature of about −60° C., a melt viscosity of about 2400 poises at 50° C. and has a weight average molecular weight of about 30,000 to about 50,000. KRATON® LVSI-101 is a material having such properties. Another material believed to have such properties is LIR-310, from Kuraray America, Pasadena, Tex.

Another example of a useful liquid rubber is a liquid polyisoprene obtained by selectively or partially degrading a high molecular weight polyisoprene. An example of a commercially available partially degraded high molecular weight polyisoprene is ISOLENE® D-400 from Elementis Performance Polymers, Belleville, N.J., and this liquid rubber has a molecular weight of about 20,000. Other liquid rubbers which may be incorporated into the adhesive mixture include liquid styrene-butadiene rubbers, liquid butadiene rubbers, ethylene-propylene rubbers, etc., as noted above.

In one embodiment, the liquid rubber component comprises a low molecular weight liquid rubber. In one embodiment, the liquid rubber component has a number average molecular weight less than about 3,000.

In one embodiment, the polyisobutylene component is a low molecular weight polyisobutylene. In one embodiment, the polyisobutylene component is exemplified by the VISTANEX® LM series of polyisobutylenes, available from ExxonMobil Chemical Corporation. In one embodiment, the polyisobutylene has a Flory viscosity average molecular weight in the range from about 25,000 to about 75,000, and in one embodiment, from about 35,000 to about 70,000, and in one from about 40,000 to about 55,000. In one embodiment, the polyisobutylene has a Brookfield viscosity at 175° C. within the range from about 10,000 to about 170,000 mPa·sec, and in one embodiment, from about 20,000 to about 140,000 mPa·sec, and in another from about 25,000 to about 70,000 mPa·sec. Brookfield viscosity is determined by measuring the shearing stress on a spindle rotating at a definite, constant speed while it is immersed in the sample. Brookfield viscosity is measured in centipoises or mPa·sec. Viscosity is a function of shear rate and is defined as shear stress/shear rate, and is measured according to ASTM D3236. In one embodiment, the Staudinger molecular weight of the low molecular weight polyisobutylenes ranges from about 5,000 to about 20,000, and in another embodiment, the Staudinger molecular weight ranges from about 10,000 to about 12,000.

In one embodiment, the low molecular weight polyisobutylene component is present in the dressing at between about 15 wt % and about 40 wt % of the total formulation, and in one embodiment, between about 20 wt % and about 30 wt % of the total formulation.

The low molecular weight polybutene components are exemplified by the HYVIS® series of materials from BP, and by the PARAPOL® series of products from ExxonMobil Chemical Company, Houston Tex., and which have molecular weights in the range 1000 to 3000, and kinematic viscosities at 100° C. within the range 180 and 3500 cSt, as measured by test method ASTM D445. Specific examples include PARAPOL® 450, 700, 950, 1300, 2400 and 2500. The commercially available PARAPOL® series of polybutene processing oils are synthetic liquid polybutenes, each individual formulation having a certain molecular weight, all formulations of which can be used in the compositions of the invention. The HYVIS® materials are believed to be marketed by BP/Amoco now under the name INDOPOL®.

Other Additives

The addition of polymer stabilizers can be advantageous, to protect an unsaturated elastomer from degradation during processing. In one embodiment, a suitable processing stabilizer is included in the continuous component. Suitable stabilizers useful in the practice of the invention include those indicated for use with styrene-olefin-styrene block copolymer thermoplastic elastomers, such as organophosphites, and the so-called hindered phenols. However, any suitable stabilizers may be employed. An example of an organophosphite stabilizer is tris(nonylphenyl) phosphite, available as POLYGARD® HR, manufactured by Uniroyal.

Particularly useful stabilizers are the hindered phenols, IRGANOX® 1010 and IRGANOX® 565, manufactured by Ciba Specialty Chemicals. IRGANOX® 1010 is believed to be benzene propanoic acid, 3,5-bis(1, 1 -dimethylethyl)-4-hydroxy-2,2-bis[[3-[3,5-bis(1,1-dimethylethyl)-4-hydroxyphenol]-1 -oxopropoxy]methyl]-1,3-propanediyl ester. IRGANOX® 565 is believed to be 4-[[4,6-bis(octylthio)-1,3,5-triazine-2-yl]amino]-2,6-bis(1,1-dimethylethyl)-phenol

Stabilizers may be used separately or in combination, and suitable ranges are within 0.3-1.5 wt % based on the total formulation. The stabilizers are generally added to the continuous phase.

Other optional ingredients such as tackifiers and plasticizers may be added to the continuous phase, to modify tack and optimize adhesion properties. Other optional ingredients such as silica and optional active ingredients such as growth factors, antimicrobial compounds and wound-healing components such as collagen may also be incorporated into the compositions of the invention.

Additional ingredients such as tackifiers and plasticizers may be added to the continuous phase, to modify tack and optimize adhesion properties. However, tackifiers used in prior art hydrocolloid adhesives may make the present composition too sticky. Thus, in one embodiment, the continuous phase is substantially free of any added tackifier. As used herein, the term “tackifier” or “tackifying resin” includes: (a) natural and modified rosins such, for example, as gum rosin, wood rosin, tall-oil rosin, distilled rosin, hydrogenated rosin, dimerized rosin, and polymerized rosin; (b) glycerol and pentaerythritol esters of natural and modified rosins, such, for example, as in the glycerol ester of pale wood rosin, the glycerol ester of hydrogenated rosin, the glycerol ester of polymerized rosin, the pentaerythritol ester of hydrogenated rosin, and the phenolic modified pentaerythritol ester of rosin; polyterpene resins having a softening point, as determined by ASTM method E28 58T, of from about 60° to 140° C., the latter polyterpene resins generally resulting from the polymerization of terpene hydrocarbons, such as the bicyclic mono-terpene known as pinene, in the presence of Freidel-Crafts catalysts at moderately low temperatures; (d) phenolic-modified terpene resins such, for example, as the resin product resulting from the condensation in an acidic medium, of a bicyclic terpene and a phenol; and (e) aliphatic petroleum hydrocarbon resins having a Ball and Ring softening point of from about 60° to about 140° C., the latter resins resulting from the polymerization of monomers consisting primarily of olefins and di-olefins. Thus, the low molecular weight polyisobutylenes disclosed for use herein are not included within the definition of tackifier as used herein. In addition, tackifying resins are usually solids at ordinary temperatures, while the low molecular weight polyisobutylenes disclosed herein are liquids at ordinary temperatures

Additional Embodiments

In one embodiment, the continuous phase is substantially free of a tackifier.

In one embodiment, the continuous phase is substantially free of added wax, mineral wax or petroleum jelly, and in one embodiment is substantially free of microcrystalline wax.

In one embodiment, the continuous phase is substantially free of copolymers such as ethylene vinyl acetate, and in one embodiment the continuous phase is substantially free of copolymers of ethylene vinyl alcohol.

In one embodiment, the continuous phase is substantially free of additives such as aloe, aloe vera extract, etc.

In one embodiment, the continuous phase is substantially free of absorbent additives such as silica, AEROSIL®, diatomaceous earth, zeolites or molecular sieve.

In one embodiment, the continuous phase is substantially free of organic solvent, particularly alcohol.

In one embodiment, the continuous phase is substantially resin-free.

In one embodiment, the continuous phase is substantially free of radiation cross-linked polymers.

In one embodiment, the dressing is free of added immune response modifier. As used herein, the term “immune response modifier” refers to a compound that possesses potent immunomodulating activity such as, for example, antiviral and/or antitumor activity, and may include compounds that modulate the production and secretion of cytokines. Various immune response modifiers are disclosed in WO 2004/080292.

In one embodiment, the dressing is free of an added medicament.

In one embodiment, the method is carried out without the use of a mechanical skin-puncturing device.

Preparation

The adhesive compositions of the invention may be prepared as follows. The solid rubber, for example a styrene-olefin-styrene copolymer, and the liquid rubber or other liquid polymer component, are blended together in a suitable mixer, normally a sigma blade mixer with an extruder discharge. The mixer is heated to about 170° C. A nitrogen flow of about 60 ml/sec through the mixer reduces the possibility of oxidative degradation of the rubber during processing. About 1 wt % of a suitable stabilizer, for example, IRGANOX® 1010 available from Ciba-Geigy, can be added at this stage. Normally a small amount of the liquid rubber, say 10-20% of the total, is added to the whole amount of the solid rubber and the liquid rubber is allowed to blend with the soft solid rubber. When all this portion of the liquid rubber has been absorbed, another portion of the liquid rubber is added, for example, another 20-30% of the total, and the liquid rubber is absorbed into the styrene-olefin-styrene rubber. This is continued until all the liquid rubber is added, when a pourable tacky intermediate adhesive is obtained. The mixer blades are stopped, the direction of the screw is reversed, and the intermediate adhesive is removed from the mixer. The adhesive is run off into suitably release coated containers and allowed to cool. The mixer is stabilized at 90° C. and the dry (e.g., powdered) ingredients are charged to the mixer. The other optional ingredients, if to be included, can be added, and blended-in for a period of time. After mixing at 90° C. for 20-30 minutes, the mixer temperature is raised to 105° C., and the ingredients of the continuous phase, intermediate hot melt and other low and high molecular weight rubbers if present, can then be added. If high molecular weight rubbers or polymers are used, they may need to be pre-masticated in the mixer, or pre-milled on a rubber mill. Mixing is continued normally for a further 30 minutes or so. The fully mixed mass is removed from the mixer and extruded or pressed to the desired thickness, after which it may be laminated to suitable substrates.

### EXAMPLES

The invention will be further illustrated by means of the following examples. Examples 1-5 correspond to the first embodiment of the continuous phase. Examples 6 and 7 correspond to the second embodiment of the continuous phase. Examples 7-16 correspond to the third embodiment of the continuous phase.

### Examples 1 and 2

Examples 1 and 2 are prepared as follows. First, an intermediate adhesive, designated as Formula 1A, is prepared:

Formula 1A

| Ingredient | wt % total | Amount, gm |
|---|---|---|
| LVSI-101 Liquid Rubber | 79.37 | 18400 |
| KRATON ® KD-1161N | 19.84 | 4600 |
| IRGANOX ® 1010 | 0.79 | 184 |
| Total | 100.00 | 23184 |

The mixture is purged with nitrogen gas and heated to 160° C. The speed of the front, faster, blade is about 30 rpm. The KRATON® KD-1161 N and the IRGANOX® 1010 are charged to the mixer at 160° C. and the mixer is started. After mixing for 5 minutes, the rubbery crumb coalesces, and 50 gm of the liquid rubber is added with continued mixing and nitrogen purging. After a further 10 minutes, the temperature is raised to 170° C. and the mixer front blade speed is increased to 47 rpm. The liquid rubber at this point is completely mixed with the rubber, and a further 51 gm of liquid rubber is added. Ten minutes later, after blending of the second portion of the liquid rubber, a further 48 gm of liquid rubber is added, and is mixed for a further 10 minutes. In this way, approximately 50 gm portions of the charge of liquid rubber are added every 10 minutes until all the 400 gm has been added. About 15 minutes later, the intermediate adhesive is removed from the mixer. The total time for this operation is about 90 minutes.

From this intermediate mixture, referred to as Formula No. 1A, two finished hydrocolloids, Examples 1 and 2, are made having the following formulae. AQUASORB® A500 is crystalline sodium carboxymethyl cellulose available from Aqualon, div. of Hercules Chemical. AEROSIL® 200 is fumed silica available from Degussa AG. Weights are in grams:

| | Example 1 | | Example 2 | |
|---|---|---|---|---|
| | Wt., gm. | Wt. % | Wt., gm. | Wt. % |
| VISTANEX ® LMMH | 65.6 | 19.4 | 65.6 | 19.6 |
| AQUASORB ® A500 | 98.5 | 29.1 | 98.5 | 29.4 |
| AEROSIL ® 200 | 10.2 | 3.0 | 6.7 | 2.0 |
| Formula 1A | 164.1 | 48.5 | 164.1 | 49.0 |
| Total | 338.4 | 100.0 | 334.9 | 100.0 |

The mixer temperature is reduced to 90° C. and the absorbent powder and silica are placed in the mixture and the mixer is started. No nitrogen purge is used in this phase of the preparation, although if desired it may be. The VISTANEX® LMMH is added, the temperature raised to 105° C. The mix is blended for 10 minutes, after which the intermediate adhesive, referred to as Formula 1A, is added. Blending is continued at 105° C. for a further 30 minutes, and the finished formulation is then removed from the mixer with a spatula. The finished dressing is pressed between two sheets of silicone release paper in a hydraulic press with the platens maintained at 90° C.

### Examples 3 and 4

Examples 3 and 4 are prepared as follows, using Formula 1A prepared as above. All weights are in grams:

| | Example 3 | | Example 4 | |
|---|---|---|---|---|
| | Wt., gm. | Wt. % | Wt., gm. | Wt. % |
| GENU ® Pectin USP-100 | 3270 | 9.33 | 0 | |
| BLANOSE ® 7H4XF | 3270 | 9.33 | 0 | |
| AQUASORB ® A500 | 3270 | 9.33 | 9810 | 28.0 |
| Formula 1A | 25200 | 72.0 | 25200 | 72.0 |
| Total | 35010 | 100.0 | 35010 | 100.0 |

BLANOSE® 7H4XF is sodium carboxymethylcellulose. The formulated adhesives are extruded at 100° C. onto a silicone coated release paper, calendered down to a gauge of 0.45 mm and laminated to an acrylic adhesive coated polyurethane film. The acrylic adhesive on the polyurethane film serves as a tie coat to anchor the absorbent adhesive to the film.

### Example 5

Example 5 is prepared as follows. First, a liquid rubber based adhesive, designated Formula 2A, is prepared as follows:

Formula 2A:

| Ingredient | Wt. % total | Amount, gm. |
|---|---|---|
| LVSI-101 Liquid rubber | 79.37 | 20000 |
| VECTOR ® 4111 | 19.84 | 5000 |
| IRGANOX ® 1010 | 0.79 | 200 |
| Total | 100.0 | 25200 |

An alternate S-I-S rubber, VECTOR® 4111, available from DEXCO, Houston, Tex., is used in order to increase shear strength, since the VECTOR® 4111 is a linear, pure SIS triblock copolymer, with narrow molecular weight distribution, a low styrene content and a low modulus, whereas the KRATON® D-1161 N contains about 17% diblock. The mixture is processed further as follows:

Using the composition of Formula 2A, the Example 5 is prepared. Weights are in grams:

| Ingredient | wt % total | Amount, gm |
|---|---|---|
| VISTANEX ® LMMH | 28 | 168 |
| GENU ® Pectin USP 100 | 14 | 84 |
| BLANOSE ® 7H4XF | 14 | 84 |
| AQUALON ® A500 | 14 | 84 |
| Formula 2A | 30 | 180 |
| Total | 100 | 600 |

### Examples 6 and 7

Examples 6 and 7, according to one embodiment of the present invention, are prepared as follows. The contents of each of Examples 6 and 7 are shown in the table following the preparation description.

A sigma blade mixer is purged with nitrogen gas and is heated to 160° C. The speed of the front, faster, blade is about 47 rpm. KRATON® KD-1161N and IRGANOX® 1010 are charged to the mixer at 160° C., and the mixer is started. After mixing for about 5 minutes, the rubbery crumb coalesces, and the mixture of tackifying agents is added with continued mixing and nitrogen purging. After the tackifiers are completely mixed with the rubber, the mixer is cooled to about 110° C. and the butyl rubber is added, together with sodium carboxymethyl cellulose. After complete mastication of the butyl rubber, the mixer is further cooled to 90° C. and the rest of the powders are added. The total time for this operation is about 90 minutes. The finished hydrocolloid is removed from the mixture with a spatula and pressed between two sheets of silicone release paper in a hydraulic press with the platens maintained at 90° C.

| | | Example 6 | | Example 7 | |
|---|---|---|---|---|---|
| Component | Description | Wt., gm. | Wt. % | Wt., gm. | Wt. % |
| KRATON ® D-1161 | S-I-S copolymer | 45.3 | 7.5 | 67.9 | 11.3 |
| ADTAC ® LV-E | C₅ resin tackifier | 23.8 | 3.95 | 35.8 | 6.0 |
| ESCOREZ ® 2203 LC | Rosin Ester | 50.0 | 8.3 | 75.0 | 12.5 |
| VISTANEX ® | Low MW PIB | 168.0 | 27.9 | 0.0 | 0 |
| LMMH | | | | | |
| VISTANEX ® LMH | Low MW PIB | 0.0 | 0 | 168.0 | 28.0 |
| IRGANOX ® 1010 | Stabilizer | 0.9 | 0.15 | 1.3 | 0.2 |
| Butyl Rubber | Modifier | 60.0 | 10.0 | 0.0 | 0 |
| AEROSIL ® 200 | Modifier | 3.0 | 0.5 | 0.0 | 0 |
| Sodium CMC | Hydrocolloid | 84.0 | 13.9 | 84.0 | 14.0 |
| GENU ® Pectin | Hydrocolloid | 84.0 | 13.9 | 84.0 | 14.9 |
| USP100 | | | | | |
| AQUASORB ® | Hydrocolloid | 84.0 | 13.9 | 84.0 | 13.9 |
| A500 | | | | | |
| TOTAL | | 603.0 | 100.0 | 600.0 | 100.0 |

### Examples 7-16

The invention is further described by the following non-limiting Examples. The following examples illustrate exemplary dressings in accordance with additional embodiments of the present invention. In the following examples, the water soluble and/or water-swellable absorbent polymer ingredients, the PIB and the mineral oil are mixed as described above, and the composition prepared in Example 1 above and referred to as Formula 1A is combined with these ingredients as described above.

The raw materials used in these examples are as follows:
LVSI-101—Kraton Polymers: Styrene-isoprene diblock copolymer (SI) liquid rubber.
KRATON® D-1161 NS—Kraton Polymers: SIS thermoplastic elastomer
IRGANOX®1010—Ciba Chemicals: Stabilizer
VISTANEX® LMMS—ExxonMobil Chemical; low molecular weight polyisobutylene
KAYDOL® Mineral Oil—Witco Chemical
GENU® Pectin USP100—CP Kelco ApS, Wilmington, Del.
GENUS Pectin LM-104 AS-FS—CP Kelco ApS, Wilmington, Del.
BLANOSE® 7H4XF—Hercules Chemical
Natrasol™ 250 CM Plus—Hercules Chemical
Natrasol™ Plus CS, Grade 330—Hercules Chemical
CARBOPOL® Ultrez 10NF—Noveon Inc.

Calcium Alginate—CP Kelco ApS, Wilmington, Del.

| | Ex. 7 | Ex. 8 | Ex. 9 | Ex. 10 | Ex. 11 | Ex. 12 | Ex. 13 | Ex. 14 | Ex. 15 | Ex. 16 |
|---|---|---|---|---|---|---|---|---|---|---|
| Ingredient | Wt % | Wt % | Wt % | Wt % | Wt % | Wt % | Wt % | Wt % | Wt % | Wt % |
| Formula 1A | 9.00 | — | 3.32 | 5.54 | 1.66 | 1.66 | 1.66 | 1.66 | 1.66 | 4.96 |
| Adhesive | | | | | | | | | | |
| VISTANEX ® | 20.00 | 23.07 | 23.07 | 23.07 | 23.07 | 23.07 | 23.07 | 23.07 | 23.07 | 22.06 |
| LMMS | | | | | | | | | | |
| GENU ® | 16.00 | 14.77 | 13.66 | 12.92 | 14.21 | 14.21 | 8.53 | 8.53 | 8.53 | 8.33 |
| Pectin | | | | | | | | | | |
| USP100 | | | | | | | | | | |
| NATRASOL ® | — | — | — | — | — | 7.11 | 8.53 | — | — | — |
| 2500CM Plus | | | | | | | | | | |
| NATRASOL ® | — | — | — | — | — | — | — | 8.53 | 8.53 | 8.33 |
| Plus CS, | | | | | | | | | | |
| Grade 330 | | | | | | | | | | |
| BLANOSE ® | 16.00 | 14.77 | 13.66 | 12.92 | 7.11 | — | 8.53 | 8.53 | 8.53 | 8.33 |
| 7H4XF | | | | | | | | | | |
| GENU ® | — | 14.77 | 13.66 | 12.92 | 14.21 | 14.21 | 8.53 | 8.53 | 8.53 | 8.33 |
| Pectin LM- | | | | | | | | | | |
| 104 AS-FS | | | | | | | | | | |
| CARBOPOL ® | — | — | — | — | — | — | — | — | 8.53 | 8.33 |
| Ultrez10NF | | | | | | | | | | |
| Calcium | — | — | — | — | 7.11 | 7.11 | 8.53 | 8.53 | — | — |
| Alginate | | | | | | | | | | |
| Mineral Oil | 39.00 | 32.63 | 32.63 | 32.63 | 32.63 | 32.63 | 32.63 | 32.63 | 32.63 | 32.33 |
| Total | 100.00 | 100.01 | 100.00 | 100.00 | 100.00 | 100.00 | 100.01 | 100.01 | 100.01 | 100.00 |

### Example 17

The following clinical example is provided, showing that treatment with a dressing in accordance with the present invention provides an improved, accelerated pigment removal from tattoo sites following laser treatment while at the same time promoting healing of the skin surface from which the tattoo is removed.

A 35-year-old male patient with a multicolored tattoo on the left upper arm is treated with laser light to remove the tattoo. The site and surrounding skin are shaved before laser treatment and then the skin is cleansed with isopropyl alcohol swabs. Although local anesthesia may not be needed, if necessary, it can be used by topical application, e.g., of EMLA® cream or by intra-dermal injection of 2% Lidocaine with Epinephrine. Before the treatment, subject's eyes are protected with goggles.

The whole tattoo is treated during the session. Depending on the colors, the appropriate lasers are used. Several lasers are used at the same treatment session, including one or more of 532 nm (Palomar Technologies, Burlington, USA), 755 nm (Candela, Wayland, USA) or 1064 nm Q-switched laser (Palomar Technologies, Burlington, USA). Repeat sessions may follow at 6-8 week intervals, as needed. In each case, the treatment is uniform over the whole tattoo area.

After the laser exposures, dressings are applied to each half of the treated area. One half is covered with Flammazine® and then is dressed with a dressing pad (Topper 8, Johnson & Johnson, Skipton, UK) and Opsite Flexifix (Smith and Nephew, Hull, UK), and the other half with the dressing of Example 4, above. No Flammazine is used under the dressing of Example 4.

After the first laser treatment the subject is required to return daily for follow-up until complete healing is achieved. After the second and third treatments, dressings may be changed by the patient at home based on verbal and written post treatment instructions.

Wound healing under the experimental dressing appears to be faster, with less redness. The patient appears to experience and reports more discomfort under the control dressing. After five completed treatment cycles, the amount of pigment remaining is observed to be less in the area covered by the dressing of Example 4 compared to that under the control dressing (Flammazineo®, Toppero® 8 and Opsite Flexifix®). Visually, tattoo removal appears more complete in the area covered by the dressing of Example 4 of the instant invention, in addition to being more comfortable to the patient.

It is noted that, throughout the specification and claims, the numerical limits of the disclosed ranges and ratios may be combined, and are deemed to include all intervening values. Furthermore, all numerical values are deemed to be preceded by the modifier “about”, whether or not this term is specifically stated.

While the principles of the invention have been explained in relation to certain particular embodiments, and are provided for purposes of illustration, it is to be understood that various modifications thereof will become apparent to those skilled in the art upon reading the specification. Therefore, it is to be understood that the invention disclosed herein is intended to cover such modifications as fall within the scope of the appended claims. The scope of the invention is limited only by the scope of the following claims.

1. A method of removing a tattoo from skin, comprising:
(a) applying laser radiation to a tattoo site; and
(b) applying to the tattoo site a dressing to absorb pigment from the tattoo site, wherein the dressing comprises a continuous phase and a discontinuous phase, the continuous phase comprising (b-1) one or more physically cross-linked solid rubber or (b-2) one or more styrene-containing thermoplastic elastomer or a mixture of (b-1) and (b-2), and the discontinuous phase comprising one or more hydrophilic polymer that is soluble and/or swellable in water.

2. The method of claim 1 further comprising repeating the steps (a) and (b) at least one additional time.

3. The method of claim 1 further comprising applying one or more of an anesthetic agent, an antibiotic agent, an anti-infective agent, or a combination of two or more thereof to the tattoo site.

4. The method of claim 1 wherein the continuous phase comprises the physically cross-linked solid rubber of (b-1), and further comprises one or more compatible liquid rubber, and one or more tackifier.

5. The method of claim 4 wherein the weight ratio of the liquid rubber to the solid rubber is from 3:2 to 7:1.

6. The method of claim 4 wherein the liquid rubber is other than polyisobutylene.

7. The method of claim 4 wherein the cross-linked solid rubber includes at least one linear or radial A-B-A block copolymer based on styrene-butadiene, styrene isoprene or hydrogenated styrene-diene copolymers.

8. The method of claim 4 wherein the cross-linked solid rubber comprises up to 85 wt %, based on the weight of the physically cross-linked solid rubber, of one or more styrene-butadiene, styrene isoprene or hydrogenated styrene-diene A-B block copolymers.

9. The method of claim 8 wherein the A-B block copolymer(s) are present in an amount of 10 to 80 wt %, based on the weight of the physically cross-linked solid rubber.

10. The method of claim 4 wherein the liquid rubber has a molecular weight of 25,000 to 50,000.

11. The method of claim 4 wherein the liquid rubber has a glass transition temperature of less than −50° C.

12. The method of claim 4 wherein the liquid rubber has a viscosity at 38° C. to 50 to 1000 Pas.

13. The method of claim 1 wherein the continuous phase comprises the physically cross-linked solid rubber of (b-1), which comprises a blend of linear or radial A-B-A block copolymers and up to 85 wt % of A-B block copolymer, based on the weight of the physically cross-linked solid rubber, a compatible tackifying resin and a low molecular weight polyisobutylene.

14. The method of claim 13 wherein the continuous phase contains from about 10 to about 30 wt % of the physically cross-linked solid rubber, based on the total weight of the continuous phase.

15. The method of claim 13 wherein the continuous phase comprises from about 18 to about 40 wt % of the compatible tackifying resin, based on the total weight of the continuous phase.

16. The method of claim 15 wherein the weight ratio of the solid rubber to the tackifying resin is from 1:0.5 to 1:7.

17. The method of claim 13 wherein the continuous phase comprises from about 10 to about 60 wt % of the low-molecular weight polyisobutylene, based on the total weight of the continuous phase.

18. The method of claim 17 wherein the low-molecular weight polyisobutylene has a viscosity average molecular weight of 30,000 to 70,000.

19. The method of claim 13 wherein the continuous phase further comprises up to about 50 wt % of modifying butyl rubber, based on the total weight of the continuous phase.

20. The method of claim 19 wherein the modifying butyl rubber has a viscosity average molecular weight of 200,000 to 600,000.

21. The method of claim 13 wherein the A-B-A block copolymer component of the cross-linked solid rubber comprises a styrene-olefin-styrene or styrene-alkane-styrene block copolymer.

22. The method of claim 13 wherein the cross-linked solid rubber comprises 15 to 50 wt % of A-B diblock copolymer, based on the weight of the physically cross-linked solid rubber.

23. The method of claim 13 wherein the A-B block copolymer component of the solid rubber comprises a styrene-butadiene, styrene isoprene or hydrogenated styrene-diene copolymer.

24. The method of claim 1 wherein the continuous phase comprises the one or more styrene-containing thermoplastic elastomer of (b-2), at least one compatible liquid rubber, at least one polyisobutylene, and at least one oil.

25. The method of claim 24 wherein the oil comprises from about 25% to about 45 wt %, based on the total weight of the dressing.

26. The method of claim 24 wherein the oil is a mineral oil.

27. The method of claim 24 wherein the weight ratio of the styrene-containing thermoplastic elastomer to liquid rubber is from about 1:0.5 to about 1:7.

28. The method of claim 24 wherein the solid rubber comprises at least one linear or radial A-B-A block copolymer based on styrene-butadiene, styrene isoprene or hydrogenated styrene-diene copolymers.

29. The method of claim 28 wherein the continuous phase comprises up to 85 wt % relative to the A-B-A block copolymer(s) of one or more styrene-butadiene, styrene isoprene or hydrogenated styrene-diene block copolymers.

30. The method of claim 24 wherein the continuous phase comprises up to about 9 wt % of the liquid rubber.

31. The method of claim 24 wherein the liquid rubber has a molecular weight in the range of about 25,000 to about 50,000.

32. The method of claim 24 wherein the liquid rubber has a viscosity at 38° C. in the range of 500 to about 10,000 poises.

33. The method of claim 24 wherein the liquid rubber comprises at least one of synthetic liquid isoprene rubber, depolymerized natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer.

34. The method of claim 24 wherein the content of the polyisobutylene is from about 15 wt % to about 40 wt % of the continuous phase.

35. The method of claim 24 wherein the polyisobutylene has a Flory viscosity average molecular weight in the range from about 25,000 to about 75,000.

36. The method of claim 24 wherein the continuous phase further comprises at least one low molecular weight polybutene.

37. The method of claim 24 wherein the weight of the combination of the styrene-containing thermoplastic elastomer and the liquid rubber is the range of about 1% to about 9%, based on the total weight of the dressing.

38. The method of claim 24 wherein the continuous phase is substantially free of a tackifier.

39. The method of claim 1 wherein the discontinuous phase comprises from about 10 wt % to about 70 wt % of the total weight of the dressing.

40. The method of claim 1 wherein the discontinuous phase comprises from about 20 wt % to about 50 wt % of the total weight of the dressing.

41. The method of claim 1 wherein the one or more hydrophilic polymer that is soluble and/or swellable in water comprises at least one water-absorbent and/or water-swellable polymer, at least one hydrocolloid, or a mixture of two or more thereof.

42. The method of claim 41 wherein the at least one water-swellable polymer comprises at least one of cross-linked sodium carboxymethyl cellulose, crystalline sodium carboxymethyl cellulose, cross-linked dextran, calcium alginate, starch-acrylonitrile graft polymer, starch sodium polyacrylate, gluten, polymers of methylvinyl ether and maleic acid and derivatives thereof.

43. The method of claim 41 wherein the at least one hydrocolloid comprises at least one of sodium carboxymethyl cellulose, pectin, gelatin, guar gum, locust bean gum, collagen, karaya gum, alginic acid, sodium alginates, sodium-calcium alginates, polyvinyl alcohol, polyvinyl pyrrolidone, polyethylene glycol and polypropylene glycol.

44. The method of claim 1 wherein the dressing is free of immune response modifier.

45. The method of claim 1 wherein the dressing is free of an added medicament.

46. The method of claim 1 wherein the method is carried out without the use of a mechanical skin-puncturing device.

47. A method of removing a tattoo from skin, comprising:
(a) applying laser radiation to a tattoo site; and
(b) applying to the tattoo site a dressing to absorb pigment from the tattoo site, wherein the dressing comprises a fluid-absorbing pressure sensitive adhesive material.

48. The method of claim 47 wherein the fluid-absorbing pressure sensitive adhesive material includes a mixture of an adhesive material and at least one hydrophilic polymer that is soluble and/or swellable in water.

49. The method of claim 48, wherein the hydrophilic polymer comprises at least one water-absorbent and/or water-swellable polymer, at least one hydrocolloid, or a mixture of two or more thereof.
